(19) United States
(12) Patent Application Publication
(10) Pub. No.: US 2011/0070664 A1
(43) Pub. Date: Mar. 24, 2011

(54) Integrated Microfluidic Device for Serum Biomarker Quantitation using Either Standard Addition or a Calibration Curve

(76) Inventors: Adam T. Woolley, Orem, UT (US); Weichun Yang, Provo, UT (US); Xiuhua Sun, Yokohama (JP)

(21) Appl. No.: 12/857,371

(22) Filed: Aug. 16, 2010

Related U.S. Application Data
(63) Continuation of application No. PCT/US2010/035333, filed on May 18, 2010.
(60) Provisional application No. 61/216,538, filed on May 18, 2009.

(51) Int. Cl. G01N 33/543; G01N 30/96; G01N 31/00; G01N 21/00; G01N 21/76; G01N 1/18; G01N 27/447

(52) U.S. Cl. 436/518; 422/69; 436/10; 436/164; 436/172; 436/178; 204/450

(57) ABSTRACT

Apparatus and method for determining concentration of one or more target compounds in a sample solution containing one or more nontarget compounds that uses an affinity column to immobilize the target compounds. The target compounds are eluted and passed through a separation/detection system to determine quantitative measure of concentration for each of the target compound.

### CROSS REFERENCE TO RELATED APPLICATIONS

This application is a continuation of Application No. PCT/US/35333, filed May 18, 2010, under the Patent Cooperation Treaty, which application claims priority from U.S. Provisional Patent Application 61/216,538, filed 18 May 2009, which all are hereby incorporated by reference.

### FEDERAL SUPPORT

This invention was made with support from United States Government, and the United States Government has certain rights in this invention pursuant to contract number R01 EB006124, National Institutes of Health.

### BACKGROUND

Biomarkers in human body fluids have great potential for use in screening for diseases such as cancer and diabetes, diagnosis, determining the effectiveness of treatments, and detecting recurrence. Present 96-well immunoassay technology effectively analyzes large numbers of samples; however, this approach is more expensive and less time effective on single or a few samples. In contrast, microfluidic systems are well suited for assaying small numbers of specimens in a point-of-care setting, provided suitable procedures are developed to work within peak capacity constraints when analyzing complex mixtures like human blood serum.

Detection and accurate quantitation of biomarkers such as alpha-fetoprotein (AFP) can be a key aspect of early stage cancer diagnosis.

Microfluidic devices provide attractive analysis capabilities, including low sample and reagent consumption, as well as short assay times. However, to date microfluidic analyzers have relied exclusively on calibration curves for sample quantitation, which can be problematic for complex mixtures such as human serum.

The two most widely used quantitation tools in traditional analytical chemistry are the calibration curve and the method of standard addition.¹ Micromachined devices for chemical analysis^{2, 3} that integrate multiple processes,⁴ reduce sample and reagent consumption,⁵ and decrease analysis time^{6, 7} and instrument footprint,^{8, 9} are becoming an attractive alternative to classical separation-based analysis approaches. Although calibration curves have been used in microchip-based chemical analysis,^{10, 11} the method of standard addition, which is especially desirable for addressing matrix effects in complex samples¹ such as blood, has seen extremely limited use. Very recently, a serial dilution microfluidic device was applied in standard addition quantitation of mM concentrations of Fe(CN)₆⁴⁻, a model analyte, although the aqueous KCl solution was not one for which matrix effects were anticipated.¹²

Due to earlier stage diagnosis and advances in cancer treatment, the five-year relative survival rate (of patients compared with controls) for all cancers has improved from 50% in 1975-1977 to 66% in 1996-2004.³⁰ Presently, cancer diagnosis is based mainly on morphological examination of a tumor biopsy, which is expensive, time consuming, and hence low in throughput.³¹ As an earlier stage tool, biomarkers can play an important role in cancer screening, diagnosis, and recurrence detection.^{32, 33} For instance, prostate-specific antigen (PSA) is a widely used analyte for prostate cancer screening.³⁴ However, an abnormal level of a single biomarker alone is not generally sufficient to diagnose cancer.³⁵ Thus, many men with PSA levels less than the 4.0 ng/mL action threshold had prostate cancer detected by biopsy (i.e., false-negatives).³⁶ Furthermore, PSA levels above 4 ng/mL are associated with other conditions such as prostatitis, reducing the specificity (i.e., false-positives).³⁴ To overcome these shortcomings, the simultaneous detection of multiple markers³⁷ would enable more sensitive and accurate cancer screening with higher throughput. For instance, Yang et al.³⁸ evaluated 12 biomarkers for gastrointestinal cancer diagnosis, and a combination of five markers significantly improved the diagnostic rate to ˜40% relative to the ˜27% rate achieved with just carcinoembryonic antigen (CEA).

Alpha-fetoprotein (AFP) is a diagnostic biomarker for Hepatocellular carcinoma (HCC),¹³ with a reported specificity of 65% to 94%.¹⁴ In general, patients with an elevated serum AFP concentration have a higher risk for HCC. Currently, enzyme linked immunosorbent assay (ELISA) is used in the clinical analysis of AFP in human serum.¹⁵ With trained personnel, ELISA can provide reliable results, although the multi-hour assay times and microplate format make ELISA best suited for clinical, rather than point-of-care (POC) diagnostics. In contrast, rapid analysis^{6, 7} and the ability to combine multiple processing steps^{4, 16} on a single device make a microfluidic-based approach very attractive for POC AFP analysis. The analysis and separation of AFP in spiked buffer solutions in a microdevice platform have been reported,¹⁷⁻¹⁹ and chip-based microfluidic assay systems for other analytes have been developed for saliva¹⁰ and blood samples.^{11, 20, 21} However, only calibration curve quantitation has been explored.

Currently, most biomarkers are detected via immunoassays such as enzyme linked immunosorbent assay (ELISA).³⁹ A recent review summarizes the advances and challenges of multiplexed immunoassay platforms.⁴⁰ However, these multi marker systems need further validation and quality control. Transferring these approaches to a microfluidic format could provide higher speed and lower reagent consumption.⁴¹ Yet, analyzing real samples in complex matrices using microdevices is challenging because the small microchip platform reduces resolving power and peak capacity relative to full-size instruments.⁴² Furthermore, due to small injected sample volumes and a short optical path, the concentration detection limit in microchips is often higher than in conventional techniques.⁴³ To overcome these shortcomings of microfluidic systems, multiple analysis functions can be integrated on a single device, enabling sample purification and preconcentration.⁴⁴ Many processing steps including sample desalting,⁴⁵ labeling,⁴⁶ and extraction⁴⁷ have been successfully performed in microchip systems. Because extraction can purify target components from complex matrices, it is an especially attractive technique for the pretreatment of real samples.

Solid phase extraction (SPE) is used heavily in sample purification. The principle of SPE is as follows: the targeted component (or components) is retained on a solid medium to separate it from the matrix, and retained materials can then be eluted for analysis. SPE has been applied successfully in a microfluidic format;^{48, 49} however, nonspecific interactions like hydrophobic absorption alone do not provide high selectivity. To circumvent this shortcoming, enzymes or antibodies can be immobilized on the solid surface.^{49, 50} For instance, pisum sativum agglutinin has been immobilized on monolithic substrates to retain glycoproteins, which can be eluted in several fractions based on their affinities.⁵¹ A recent review summarizes the application of immunoaffinity capillary electrophoresis (CE) for biomarker, drug and metabolite analysis in biological samples.⁵² These studies indicate a promising future for immunoaffinity extraction as a pretreatment method for biological specimens in microdevices.

### REFERENCES

(1) Skoog, D. A.; Holler, F. J.; Crouch, S. R., Eds. Principles of Instrumental Analysis; 6^{th} Ed. Thomson/Brooks Cole: Belmont, 2007.
(2) DeMello, A. J. Nature 2006, 442, 394-402.
(3) Wheeler, A. R. Science 2008, 322, 539-540.
(4) Toriello, N. M.; Liu, C. N.; Blazej, R. G.; Thaitrong, N.; Mathies, R. A. Anal. Chem. 2007, 79, 8549-8556.
(5) Murphy, B. M.; He, X.; Dandy, D.; Henry, C. S. Anal. Chem. 2008, 80, 444-450.
(6) Jacobson, S. C.; Culbertson, C. T.; Daler, J. E.; Ramsey, J. M. Anal. Chem. 1998, 70, 3476-3480.
(7) McClain, M. A.; Culbertson, C. T.; Jacobson, S. C.; Allbritton, N. L.; Sims, C. E.; Ramsey, J. M. Anal. Chem. 2003, 75, 5646-5655.
(8) Blazej, R. G.; Kumaresan, P.; Mathies, R. A. Proc. Natl. Acad. Sci. USA 2006, 103, 7240-7245.
(9) Kumaresan, P.; Yang, C. J.; Cronier, S. A.; Blazej, R. G.; Mathies, R. A. Anal. Chem. 2008, 80, 3522-3529.
(10) Herr, A. E.; Hatch, A. V.; Throckmorton, D. J.; Tran, H. M.; Brennan, J. S.; Giannobile, W. V.; Singh, A. K. Proc. Natl. Acad. Sci. USA 2007, 104, 5268-5273.
(11) Fan, R.; Vermesh, O.; Srivastava, A.; Yen, B. K.; Qin, L.; Ahmad, H.; Kwong, G. A.; Liu, C. C.; Gould, J.; Hood, L.; Heath, J. R. Nat. Biotechnol. 2008, 26, 1373-1378.
(12) Stephan, K.; Pittet, P.; Sigaud, M.; Renaud, L.; Vittori, O.; Morin, P.; Ouaini, N.; Ferrigno, R. Analyst 2009, 134, 472-477.
(13) Wright, L. M.; Kreikemeier, J. T.; Fimmel, C. J. Cancer Detect. Prev. 2007, 31, 35-44.
(14) Zinkin, N. T.; Grall, F.; Bhaskar, K.; Otu, H. H.; Spentzos, D.; Kalmowitz, B.; Wells, M.; Guerrero, M.; Asara, J. M.; Libermann, T. A.; Afdhal, N. H. Clin. Cancer Res. 2008, 14, 470-477.
(15) Debruyne, E. N.; Delanghe, J. R. Clin. Chim. Acta 2008, 395, 19-26.
(16) Wen, J.; Guillo, C.; Ferrance, J. P.; Landers, J. P. Anal. Chem. 2007, 79, 6135-6142.
(17) Kawabata, T.; Watanabe, M.; Nakamura, K.; Satomura, S. Anal. Chem. 2005, 77, 5579-5582.
(18) Bharadwaj, R.; Park, C. C.; Kazakova, I.; Xu, H.; Paschkewitz, J. S. Anal. Chem. 2008, 80, 129-134.
(19) Kawabata, T.; Wada, H. G.; Watanabe, M.; Satomura, S. Electrophoresis 2008, 29, 1399-1406.
(20) Wen, J.; Guillo, C.; Ferrance, J. P.; Landers, J. P. J. Chromatogr. A 2007, 1171, 29-36.
(21) Peoples, M. C.; Karnes, H. T. Anal. Chem. 2008, 80, 3853-3858.
(22) Dodge, A.; Fluri, K.; Verpoorte, E.; de Rooij, N. F. Anal. Chem. 2001, 73, 3400-3409.
(23) Sun, X.; Yang, W.; Pan, T.; Woolley, A. T. Anal. Chem. 2008, 80, 5126-5130.
(24) Sterling, R. K.; Jeffers, L.; Gordon, F.; Sherman, M.; Venook, A. P.; Reddy, K. R.; Satomura, S.; Schwartz, M. E. Am. J. Gastroenterol. 2007, 102, 2196-2205.
(25) Yang, W.; Sun, X.; Pan, T.; Woolley, A. T. Electrophoresis 2008, 29, 3429-3435.
(26) Kelly, R. T.; Woolley, A. T. Anal. Chem. 2003, 75, 1941-1945.
(27) Lagally, E. T.; Scherer, J. R.; Blazej, R. G.; Toriello, N. M.; Diep, B. A.; Ramchandani, M.; Sensabaugh, G. F.; Riley, L. W.; Mathies, R. A. Anal. Chem. 2004, 76, 3162-3170.
(28) Tsukagoshi, K.; Jinno, N.; Nakajima, R. Anal. Chem. 2005, 77, 1684-1688.
(29) Trevisani, F.; D'Intino, P. E.; Morselli-Labate, A. M.; Mazzella, G.; Accogli, E.; Caraceni, P.; Domenicali, M.; De Notariis, S.; Roda, E.; Bernardi, M. J. Hepatol. 2001, 34, 570-575.
30. American Cancer Society, Cancer Facts and Figures 2009. http://www.cancer.org/downloads/STT/500809web.pdf (Access date: Mar. 23, 2010)
31. S. J. Nass and H. L. Moses, eds., Cancer biomarkers: the promises and challenges of improving detection and treatment, National Academies Press, Washington, D.C., 2007.
32. N. B. Kiviat and C. W. Critchlow, Dis. Markers, 2002, 18, 73-81.
33. M. Verma, D. Seminara, F. J. Arena, C. John, K. Iwamoto and V. Hartmuller, Mol. Diagn. Ther., 2006, 10, 1-15.
34. D. V. Makarov, S. Loeb, R. H. Getzenberg and A. W. Partin, Annu. Rev. Med., 2009, 60, 139-151.
35. G. A. Sinise, ed., Tumor markers research perspectives, Nova Science Publishers, New York, 2007.
36. I. M. Thompson, D. K. Pauler, P. J. Goodman, C. M. Tangen, M. S. Lucia, H. L. Parnes, L. M. Minasian, L. G. Ford, S. M. Lippman, E. D. Crawford, J. J. Crowley and C. A. Coltman, Jr., N. Eng. J. Med., 2004, 350, 2239-2246.
37. E. Sunami, M. Shinozaki, C. S. Higano, R. Wollman, T. B. Dorff, S. J. Tucker, S. R. Martinez, F. R. Singer and D. S. B. Hoon, Clin. Chem., 2009, 55, 559-567.
38. X.-Q. Yang, L. Yan, C. Chen, J.-X. Hou and Y. Li, Hepatogastroenterology, 2009, 56, 1388-1394.
39. M. H. Bronchud, M. Foote, G. Giaccone, O. Olopade and P. Workman, eds., Principles of molecular oncology, Humana Press, Totowa, N.J., 2^{nd} Ed., 2004.
40. A. A. Ellington, I. J. Kullo, K. R. Bailey and G. G. Klee, Clin. Chem., 2010, 56, 186-193.
41. P. S. Dittrich, K. Tachikawa and A. Manz, Anal. Chem., 2006, 78, 3887-3907.
42. R. Bharadwaj, J. G. Santiago and B. Mohammadi, Electrophoresis, 2002, 23, 2729-2744.
43. W. Yang and A. T. Woolley, J. Assoc. Lab. Autom., 2010, in press.
44. E. T. Lagally, C. A. Emrich and R. A. Mathies, Lab Chip, 2001, 1, 102-107.
45. N. Lion, V. Gobry, H. Jensen, J. S. Rossier and H. Girault, Electrophoresis, 2002, 23, 3583-3588.
46. M. Yu, H.-Y. Wang and A. T. Woolley, Electrophoresis, 2009, 30, 4230-4236.
47. W. Yang, X. Sun, H.-Y. Wang and A. T. Woolley, Anal. Chem., 2009, 81, 8230-8235.
48. J. Wen, C. Guillo, J. P. Ferrance and J. P. Landers, Anal. Chem., 2007, 79, 6135-6142.
49. W. Yang, X. Sun, T. Pan and A. T. Woolley, Electrophoresis, 2008, 29, 3429-3435.
50. T. M. Phillips and E. F. Wellner, Electrophoresis, 2009, 30, 2307-2312.
51. X. Mao, Y. Luo, Z. Dai, K. Wang, Y. Du and B. Lin, Anal. Chem., 2004, 76, 6941-6947.
52. N. A. Guzman, T. Blanc and T. M. Phillips, Electrophoresis, 2008, 29, 3259-3278.
53. L. M. Wright, J. T. Kreikemeier and C. J. Fimmel, Cancer Detect. Prev., 2007, 31, 35-44.
54. Y.-A. Park, K. Y. Lee, N. K. Kim, S. H. Baik, S. K. Sohn and C. W. Cho, Ann. Surg. Oncol., 2006, 13, 645-650.
55. K. Barczyk, M. Kreuter, J. Pryjma, E. P. Booy, S. Maddika, S. Ghavami, W. E. Berdel, J. Roth and M. Los, Int. J. Cancer, 2005, 116, 167-173.
56. D. Mahalingam, R. Swords, J. S. Carew, S. T. Nawrocki, K. Bhalla and F. J. Giles, Br. J. Cancer, 2009, 100, 1523-1529.
57. N.Y. Lee, Y. Yang, Y. S. Kim and S. Park, Bull. Korean Chem. Soc., 2006, 27, 479-483.
58. B. M. Paegel, L. D. Hutt, P. C. Simpson and R. A. Mathies, Anal. Chem., 2000, 72, 3030-3037.
59. E. T. Lagally, J. R. Scherer, R. G. Blazej, N. M. Toriello, B. A. Diep, M. Ramchandani, G. F. Sensabaugh, L. W. Riley and R. A. Mathies, Anal. Chem., 2004, 76, 3162-3170.
60. R. T. Kelly and A. T. Woolley, Anal. Chem., 2003, 75, 1941-1945.
61. X. Sun, W. Yang, T. Pan and A. T. Woolley, Anal. Chem., 2008, 80, 5126-5130.

### SUMMARY

An aspect of the invention is a method for determining concentration of target compounds in solutions containing one or more target compounds and one or more nontarget compounds;

(a) passing a sample of the solution through an affinity column, the affinity column having a surface with immobilizing sites for the target compound, so that as the sample is passed through the column target compound is immobilized on the surface, allowing nontarget compounds to remain in solution as it is passed through and out from the column;
(b) eluting the target compounds by passing an eluting buffer solution through the affinity column to produce a target solution containing target compounds without nontarget compounds that were in the organic solution sample,
(c) passing the target solution through a separation column coupled with a detector to produce a quantitative measure of concentration for each of the target compounds.

The passing of the solution through an affinity column and eluting immobilized target compound, as in (a) and (b) are to produce a solution with proportional concentrations of target compounds, with low or no concentrations of nontarget compounds that may interfere with the detection and quantitative measure of target compounds. Such interference by non-target compounds can come from production of competing detection peaks or noise, making detection of peaks for the target compounds difficult.

This is accomplished by first removing target compounds from the sample solution by immobilization, and then discarding the remaining portion of the sample solution that contains nontarget compounds. An new solution comprising the target compounds, that is essentially free of non-target interfering compounds, is then created by eluting the immobilized target compounds into a solution. Without the non-target compounds in the solution, interference that might interfere with quantification of target compounds is minimized, and as illustrated examples below, may by essentially eliminated.

In this context, target compounds are compounds, organic or nonorganic, for which it is wished to determine a quantitative measure of concentration in a sample solution, such as for example, blood serum. Non-target compounds are compounds in sufficient concentration in the sample to interfere with this determination of quantitative measure. The quantitative measure of concentration is a value proportional to the concentration, and is in any arbitrary unit, or as the output value of the detector (i.e., voltage). The actual concentration of target compounds in a sample can be determined by comparison of quantitative measures of the sample with unknown concentration and quantitative measures of comparative samples, which can be, for example, calibration samples of known concentration or standard addition samples. It is also contemplated that the sample solution and the buffer solution contain other substances that do not materially interfere with the determination of a quantitative measure.

The affinity column is a column that has a surface with immobilization sites designed to reversibly immobilize target compounds on the surface. The nature of the immobilization sites depends on the properties of the target- and non-target compounds. As an example, the immobilization sites may be may antibodies where the target-compounds or compound is a antigen. In like manner, the immobilization site may include hosts, for formation of a host-guest complex with a guest target-compound (such as macrocycles). The immobilization sites may also include aptamers. The immobilization sites may be the same or comprise several types, depending upon the number of target-compounds.

The affinity column may take the form of a microchannel in a microdevice. The surface can then be treated to apply immobilization sites. For example, a thin film of a reactive polymer can be photopolymerized on the surface and antibodies, or other immobilization sites, covalently attached. The surface may be the surface of the microchannel, or comprise the surfaces of a monolith or packed bead in the column.

The eluting buffer solution is designed to reverse the immobilization reaction on the affinity column surface and release immobilized target-compound into the buffer solution. The exact composition of the buffer is designed according to the immobilization sites present.

The separation column is designed to separate and thereby quantify target compounds passing through the column. The separation column may be based upon any suitable separation scheme or system, such as capillary electrophoresis systems, optical systems, electrochemical systems, chemiluminescence systems, and absorbance systems. The detector is used to detect the and measure the relative heights of concentration peaks of compounds leaving the separation column. Because the solution going through the separation column is the eluted buffer solution containing target compounds without interfering non-target compounds, the peaks of the target compounds are easier to resolve.

The method may be conducted with structure in the form of a microdevice. Micro channels can be formed in suitable substrates that convey one or more sample solutions in succession to an microchannel affinity column. Microchannels are provided to convey the buffer solution through the affinity column and to the separation column, which can be a microchannel. The solutions can be passed through the microchannels and components of the microdevice by electrophoresis, or be pressure driven or electrically driven.

The detector for detecting amplitude of concentration peaks can be any known system, particularly those applicable to microdevices. These include systems based upon fluorescently tagged target molecules, electrochemical systems, chemiluminescence systems, and absorbance systems.

Another aspect of the present invention is an integrated microdevice comprising:

an affinity column in the form of a microchannel having a surface with immobilizing sites;

structure including channels for selectively directing multiple sample solutions from multiple sample solution sources or reservoirs to and through the affinity column;

the immobilizing sites chosen to immobilize one or more target compounds in a solution passing through the affinity column,

structure including channels for discarding solution passed through the column and after target compounds have been immobilized on the column surface

structure including channels for eluting the affinity column by passing an eluting solution through the column;

structure including one or more separation channels for detecting a quantitative concentrations target compounds in the solution eluted from the affinity column

structure including channels for directing eluted solution from the affinity column to the structure for the detecting structure.

The structure for directing multiple sample solution from multiple sample solutions sources to and through the affinity column can be any suitable structure, including, for example, microchannels, capillaries, and the like. Likewise the structure including channels for discarding solution passed through the column, and structure including channels for eluting the affinity column can be any suitable construction such as microchannels, capillaries, and the like.

### BRIEF DESCRIPTION OF DRAWINGS

FIG. 1. Immunoaffinity extraction overview.

FIG. 2. Layout of an exemplary integrated AFP analysis microchip. (a) Diagram and (b) photograph of a microfluidic device with integrated affinity column. Reservoir labels are A: sample, B: rinse buffer, C: elution solution, D: 5 ng/mL AFP standard solution, E: 10 ng/mL AFP standard solution, F: 20 ng/mL AFP standard solution, G: 5 mM NaOH (to neutralize the acidic elution solution during injection), H: waste, and I-L: electrophoresis buffer. Scale bar in (b) is 1 cm.

FIG. 3. Schematic diagram of operation of the exemplary microchip with integrated affinity column. (a) Sample loading, (b) standard loading, (c) rinsing, (d) injection, and (e) separation.

FIG. 4. Graph showing microchip electrophoresis of a mixture (a) before and (b) after affinity column extractions. Peaks are FITC-Gly, GFP, FITC-BSA, FITC-AFP, and FITC-IgG respectively. The y axis scale is the same in both (a) and (b).

FIG. 5. Graph showing FITC-labeled human serum, run by microchip electrophoresis (a) before and (b) after integrated affinity column extraction.

FIG. 6. Integrated calibration curve and standard addition quantification of AFP in human serum. (a) Microchip electrophoresis of Alexa Fluor 488 labeled human serum and of AFP standard solutions after affinity column extraction. Curves in order are: black—unknown human serum sample, red—5 ng/mL standard AFP, green—10 ng/mL standard AFP, and blue—20 ng/mL standard AFP. (b) Microchip electrophoresis of Alexa Fluor 488 labeled human serum after standard addition and affinity column extraction. Traces are: black—sample, red—sample+5 ng/mL standard AFP, green—sample+10 ng/mL standard AFP, and blue—sample+20 ng/mL standard AFP. (c) Calibration curve generated from (a), with unknown sample data point indicated with a star. (d) Standard addition plot of concentration of standard added vs. peak height generated from (b).

FIG. 7. Accuracy and precision data for integrated microfluidic AFP assay. Red: spiked concentration, green: measured by ELISA (United Biotech, Mountain View, Calif.), blue: measured by calibration curve, and light blue: measured by standard addition. Error bars indicate ±one standard deviation.

FIG. 8. Layout of an exemplary integrated microdevice. (a) Schematic diagram and (b) photograph of a typical microchip with integrated affinity column. See the text for reservoir numbering.

FIG. 9. Background-subtracted fluorescence signal on a typical affinity column after washing, for multiple AFP concentrations. The lower concentration points are expanded in the inset.

FIG. 10. Fluorescence signal from the affinity column during loading and rinsing steps. All points are average values from CCD images, and standard deviations (not shown, ˜200 units) were calculated from ˜32,000 pixels in the CCD images. The relative standard deviation values reflect some heterogeneity in the density of immobilized antibodies on the column, as well as minor imperfections on the PMMA surfaces from device bonding.

FIG. 11. The relationship between background subtracted CCD signal and the concentration of fluorescently labeled proteins. Error bars indicate standard deviations (n=3).

FIG. 12. The amounts of retained proteins on the affinity columns in three different microdevices. Standard deviations were calculated from the regression data in FIG. 11.

FIG. 13. Alexa Flour 488-labeled biomarker mixture (1 μg/mL for each protein), run by microchip electrophoresis (a) before and (b) after integrated affinity column extraction.

FIG. 14. Microchip CE of Alexa Fluor 488-labeled human serum and of standard solutions after affinity column extraction. Curves are: black—unknown spiked human serum sample, red—5 ng/mL standard mixture, green—10 ng/mL standard mixture, and blue—20 ng/mL standard mixture.

FIG. 15. Microchip electrophoresis of Alexa Fluor 488-labeled human serum after standard addition and affinity column extraction. Curves are: black—unknown spiked human serum sample, red—serum sample+5 ng/mL standard mixture, green—serum sample+10 ng/mL standard mixture, and blue—serum sample+20 ng/mL standard mixture.

### DETAILED DESCRIPTION

At aspect of the invention involves integrated microdevices with an affinity column and capillary electrophoresis channels to isolate and quantitate a panel of proteins in complex matrices. In an specific embodiment, an affinity column was formed, by photopolymerizing a thin film of a reactive polymer in a microchannel, and covalently immobilizing to it multiple (in this embodiment four) antibodies. The retained protein amounts were consistent from chip to chip, demonstrating reproducibility. Furthermore, the signals from four fluorescently labeled proteins captured on-column were in the same range after rinsing, indicating the column has little bias toward any of the four antibodies or their antigens.

These affinity columns have been integrated with capillary electrophoresis separation, enabling simultaneous quantification of multiple protein biomarkers in human blood serum in the low ng/mL range using either a calibration curve or standard addition. These systems provide a fast, integrated and automated platform for multiple biomarker quantitation in complex media such as human blood serum.

In another aspect is an integrated microfluidic system that couples immunoaffinity extraction with rapid microchip capillary electrophoresis (CE) separation for quantitation of alpha-fetoprotein (AFP) in human blood serum, using either standard addition or a calibration curve for determining concentrations.⁴⁷

Another aspect is the fabrication of integrated polymer microfluidic systems that can quantitatively determine fluorescently labeled AFP in human serum, using either the method of standard addition or a calibration curve. The microdevices couple an immunoaffinity purification step with rapid microchip electrophoresis separation with laser-induced fluorescence detection system, all under automated voltage control in a miniaturized polymer microchip. In conjunction with laser-induced fluorescence detection, these systems can quantify AFP at ˜1 ng/mL levels in ˜10 L of human serum in a few tens of minutes. The polymer microdevices have been applied in determining AFP in spiked serum samples. These integrated microsystems offer excellent potential for rapid, simple and accurate biomarker quantitation in a point-of-care setting.

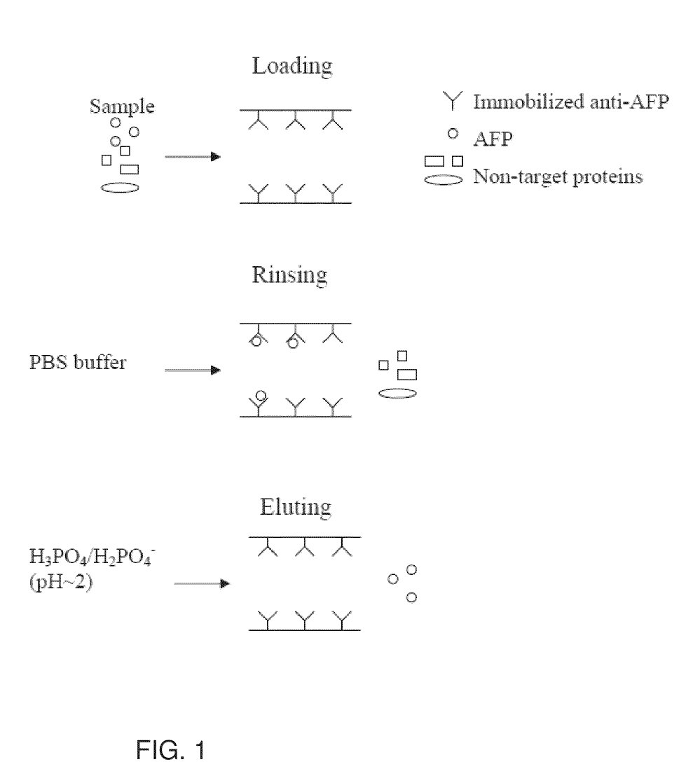

Another aspect is a microfluidic immunoaffinity extraction, which is illustrated in FIG. 1. Antibodies are immobilized on a patterned section of a microchannel surface to form an affinity column. When a sample flows through the column, only antigen will be retained based on antibody-antigen interaction, while non-target material will pass through the column to waste. This approach has been shown to capture target proteins from buffer solutions²² in a microdevice. The current system has the ability to work with complex specimens such as blood, and integrate capture with separation²³.

In a specific example is demonstrated an integrated microfluidic system capable of performing quantitative determination of AFP, a biomarker for liver cancer,²⁴ in human serum, using both the method of standard addition and a calibration curve. This approach utilizes an immunoaffinity purification step coupled with rapid microchip electrophoresis separation, all under voltage control, in a miniaturized polymer microchip. These systems with laser-induced fluorescence (LIF) detection can quantify AFP at ˜1 ng/mL levels in ˜10 μL of human serum in a few tens of minutes, offering exciting potential for POC applications.

Another aspect is an integrated microfluidic system that can simultaneously quantify multiple cancer biomarkers in human blood serum. To demonstrate this aspect commercially available biomarkers as test proteins were selected (Table 2).⁵³⁻⁵⁶ Antibodies were attached to microchip columns, and the amounts of immobilized antibodies were characterized. Integrated microdevices to quantify these four proteins at low ng/mL levels, which are in the range of their action thresholds in human blood serum. These results demonstrate that the platform is generalizable and applicable for the simultaneous quantification of multiple biomarkers in complex samples.

### Example I

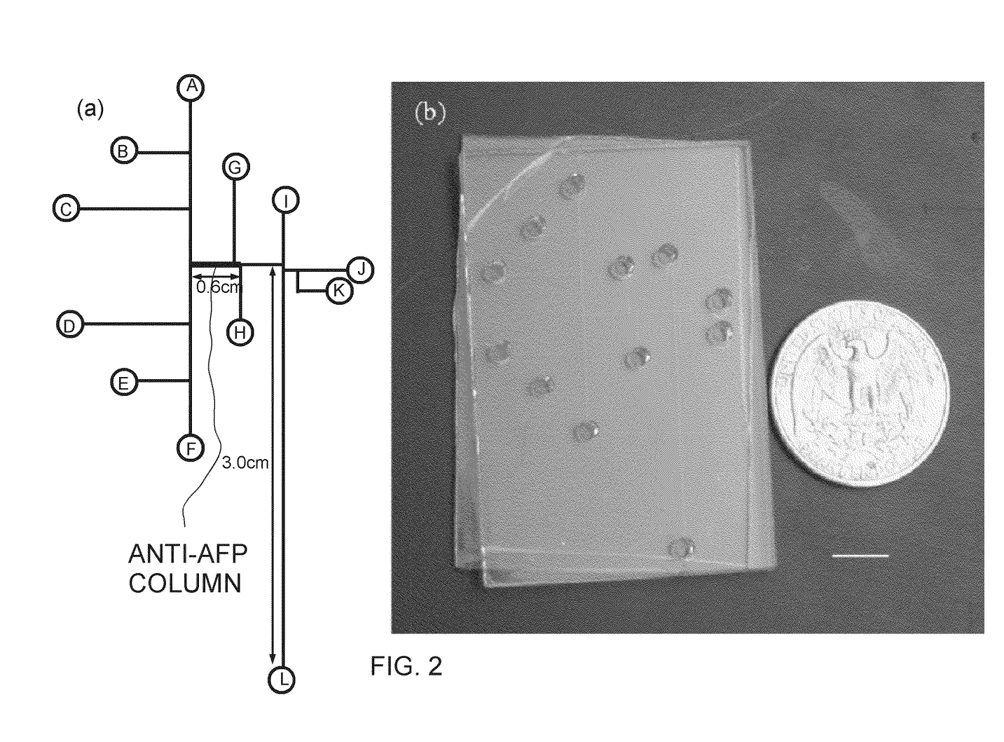

Affinity column formation. A prepolymer mixture containing glycidyl methacrylate as the functional monomer, poly(ethylene glycol) diacrylate (575 Da average molecular weight) as the crosslinker, and 2,2-dimethoxy-2-phenylacetophenone as the photoinitiator was prepared. Before polymerization, the mixture was sonicated in a water bath for 1 min, followed by nitrogen purging for 3 min to remove dissolved oxygen. The degassed mixture (10 μL) was pipetted into reservoir G (FIG. 2a), filling the microchannel via capillary action. Next, vacuum was applied to reservoir G to remove most of the monomer solution, leaving a coating of the prepolymer mixture on the channel walls. The microchip was covered with an aluminum photomask with a 4×4 mm² opening to provide spatial control of polymerization. The microchip was then placed on a copper plate in an icebath, and exposed to UV light (200 mW/cm²) in the wavelength range of 320-390 nm for 5 min (cooling helped minimize undesired thermal polymerization). Finally, any unpolymerized material was removed by flushing 2-propanol through the microchannels using a syringe pump.

Fluorescently tagged sample preparation. A 3-mL aliquot of fresh human blood was obtained from a healthy volunteer in a 4-mL Vacutainer tube (BD) at the Brigham Young University Student Health Center. The blood sample was centrifuged at 5,000 rpm (Eppendorf 5415C) for 10 min to separate the serum from whole blood. FITC and Alexa Fluor 488 TFP Ester (Invitrogen) were used to label amino acids, proteins, and serum samples using protocols provided by Invitrogen (MP 00143). Briefly, 0.1 mg fluorescent dye was dissolved in 10 μL DMSO. For amino acid or protein standards, a 5-μL aliquot of this DMSO solution was immediately mixed with 0.2 mL of sample (1 mg/mL) in 10 mM carbonate buffer (pH 9.0). For serum samples, a 2-μL aliquot of DMSO solution with dissolved dye was mixed directly with 98 μL of human serum. The mixture was incubated in the dark at room temperature for 24 h (FITC) or 15 min (Alexa Fluor 488). In direct labeling of complex biological specimens, it is essential to have excess dye to ensure complete labeling.

Data analysis. The calculation of AFP concentration was based on the peak heights in the electropherograms both for calibration curve and standard addition methods. For the calibration curve, the AFP peak height from each standard electropherogram was plotted against the AFP standard concentration to generate a linear calibration curve by the method of least squares. The AFP concentration in the sample was obtained from the electropherogram peak height and the calibration curve. The standard addition method, which effectively eliminates matrix effects,¹ was also used to analyze the AFP samples. Indeed, the present protocol of loading sample plus standard on the affinity column is microfluidically equivalent to spiking standards into a sample in a classical standard addition analysis. Peak heights from the electropherograms of the unknown sample, as well as those of the sample plus added standard, were plotted vs. concentration of added standard. The slope and intercept of this line were calculated by least squares analysis, and the unknown AFP concentration was given by the intercept divided by the slope.¹ Standard deviations were calculated from the regression data.

### RESULTS AND DISCUSSION

Used was a photo-defined immunoaffinity column in a polymeric microdevice to extract AFP from blood serum. Retained AFP was eluted through an injection cross and rapidly analyzed by microchip electrophoresis. To quantify the serum AFP concentration precisely, both standard addition and calibration curve functions were integrated into the chip. Importantly, all fluid control on-chip was carried out via voltages applied to reservoirs, facilitating automation. The fabrication protocol for poly(methyl methacrylate) (PMMA) microdevices, which entailed hot embossing and thermal annealing, was adapted from previous work.^{23, 25} The layout of the integrated AFP analysis microchip is shown in FIG. 2a, and a device photograph can be seen in FIG. 2b. PMMA itself is relatively inert toward direct chemical reaction, which necessitates making a photo-defined polymer on the microchannel surface to immobilize antibodies. The thickness of the reactive polymer formed on the channel surface was ˜3 μm. To provide analyte specificity, reactive polymer coated microchannels were derivatized with monoclonal anti-AFP according to a previously described procedure.²³

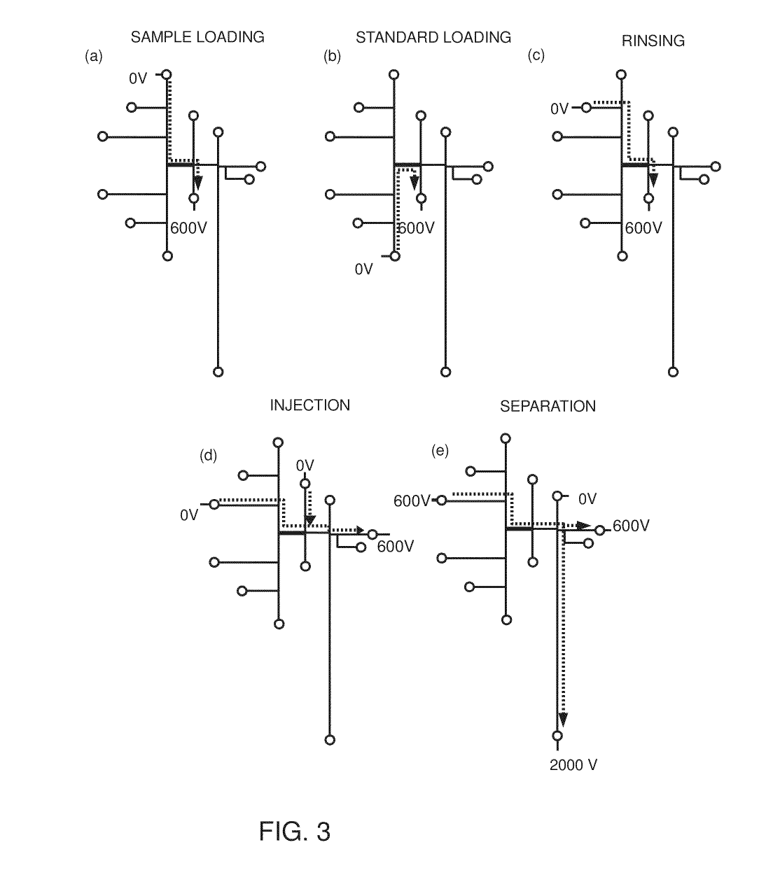

To quantify the AFP concentration in serum samples, both calibration curve and standard addition methods were used to validate the accuracy and precision of microchip performance. The voltage configurations and flow paths during operation of the microchip (described below) are shown in FIG. 3. For the calibration curve, each AFP standard solution was loaded on the affinity column for 5 min by applying voltage between either reservoir D, E, or F and reservoir H; the column was rinsed with PBS buffer for 5 min by applying a potential between reservoirs B and H; analyte was eluted/injected with a voltage applied to reservoir J while grounding reservoirs C and G for 45 s using phosphoric acid/dihydrogen phosphate solution at pH 2.1; and then loaded material was separated by microchip electrophoresis using a potential between reservoirs I and L. The sample was analyzed by loading on the affinity column for 5 min with voltage applied between reservoirs A and H, and then rinsing, elution/injection and separation were done the same as with the standards. For the standard addition method, after loading sample on the affinity column for 5 min as above, one standard was loaded on the affinity column for 5 min as before, followed by rinsing, elution/injection and microchip electrophoresis separation, the same as for the calibration curve. This process was then repeated for each standard. LIF was used to detect the labeled AFP during microchip electrophoresis.²⁶ Miniaturized (shoebox size) LIF systems for microchip electrophoresis have been made,²⁷ indicating their suitability for POC assays.

The loading, rinsing, and elution profile of fluorescein-5-isothiocyanate (FITC) labeled AFP flowing out from an anti-AFP column was characterized. A fluorescence video image taken after the end of the affinity column shows the retention, rinsing, and elution steps for FITC-AFP.

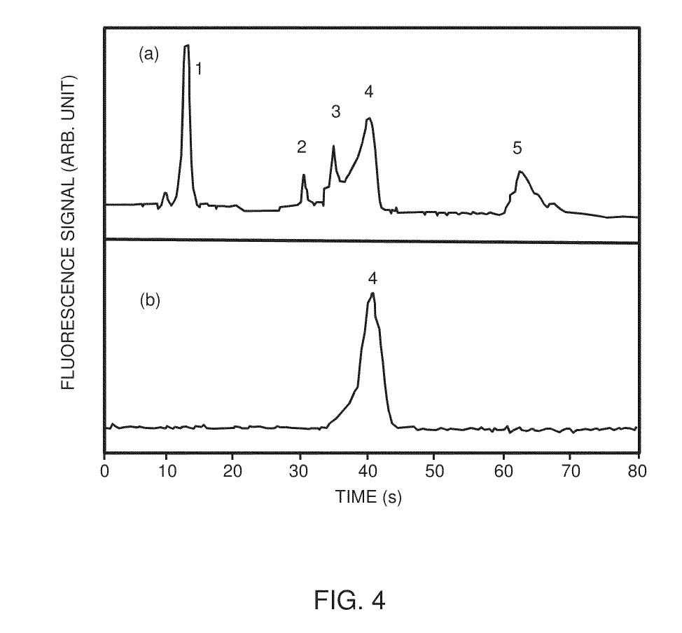

To demonstrate the integration of immunoaffinity extraction with microchip electrophoresis on a microdevice, a mixture of non-target fluorescent compounds along with FITC-AFP was loaded through an affinity column and then analyzed. Five peaks were observed before extraction, as shown in FIG. 4a. Note that FITC-BSA and FITC-AFP have similar elution times, and are not baseline resolved in the electropherogram. Contrastingly, after on-chip affinity purification (FIG. 4b), all non-target peaks are essentially eliminated, while only the AFP peak remains.

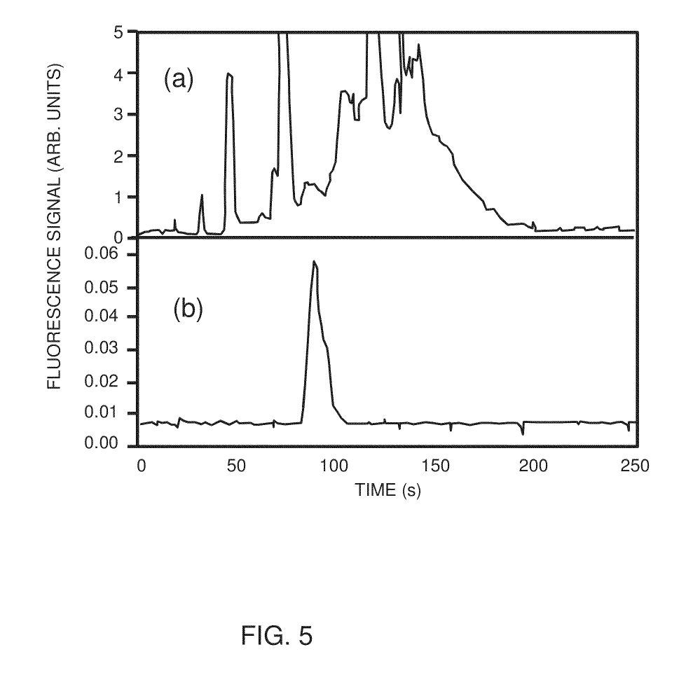

Importantly, similar device performance was observed with a much more complex, fluorescently labeled human serum sample. Microchip electrophoresis of FITC-tagged human serum (FIG. 5a) showed numerous overlapping peaks before extraction, precluding facile AFP determination. On the other hand, after on-chip AFP extraction, a single, clear peak corresponding to AFP was observed in microchip electrophoresis (FIG. 5b). The integrated immunoaffinity extraction step resulted in a ˜5,000-fold reduction of non-target protein signal, and enabled detection of the AFP “needle” in the serum “haystack”. It was estimated that the AFP sample is >95% pure after immunoaffinity extraction, based on target to spurious peak ratios in the electropherograms in FIGS. 4-5. These results clearly indicate that this approach can selectively purify target analytes from very complex mixtures. A typical affinity column can perform well for at least a few tens of replicate runs.

FITC is a commonly used fluorescent dye for labeling amine-containing compounds such as proteins; however, the room-temperature reaction kinetics (˜24 h), make this label less desirable for POC work. On the other hand, it was found that Alexa Fluor 488 TFP Ester (Invitrogen) completely labeled AFP in ˜30 min, making this dye very well suited for POC work. In addition, for some microchip bioassays, sample and standards share the same reservoir,^{10, 28} requiring a cleaning step during analysis, which hampers the ability to automate for POC assays. In this design, sample and standard reservoirs are integrated on the microdevices. Finally, although previous systems have only used calibration curves to quantify biomarkers,^{10, 11} This format enables both standard addition and calibration curve protocols to be performed on-chip.

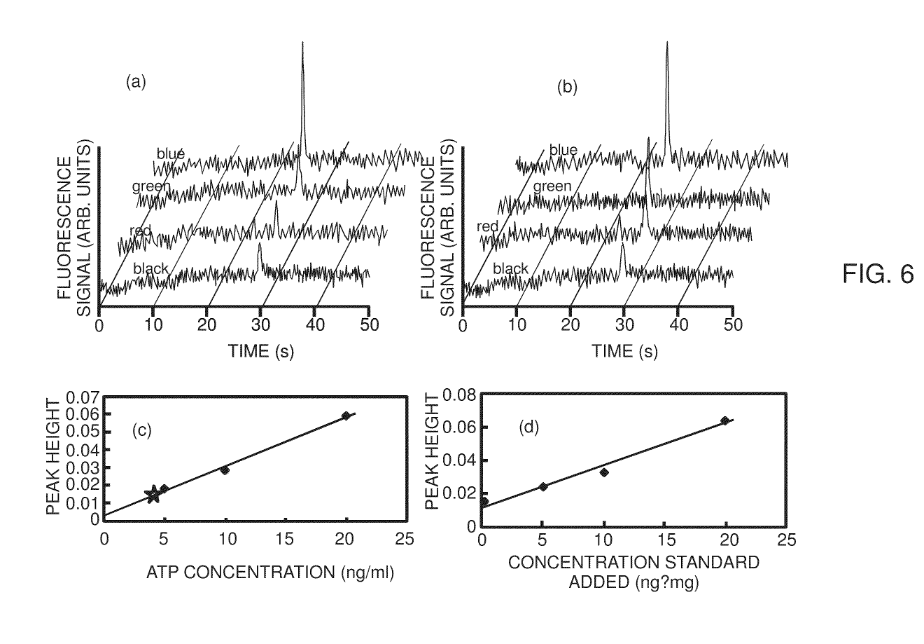

The integrated microdevices were used to quantify AFP concentration in human serum using either a linear calibration curve (FIG. 6a, 6c) or the standard addition method (FIG. 6b, 6d). Both approaches yielded reproducible microchip electrophoresis data (FIG. 6a, 6b) with concentration-dependent peak heights (FIG. 6c, 6d). AFP concentrations and standard deviations determined both by calibration curve (4.1±0.9 ng/mL) and standard addition methods (4.6±0.9 ng/mL) were internally consistent.

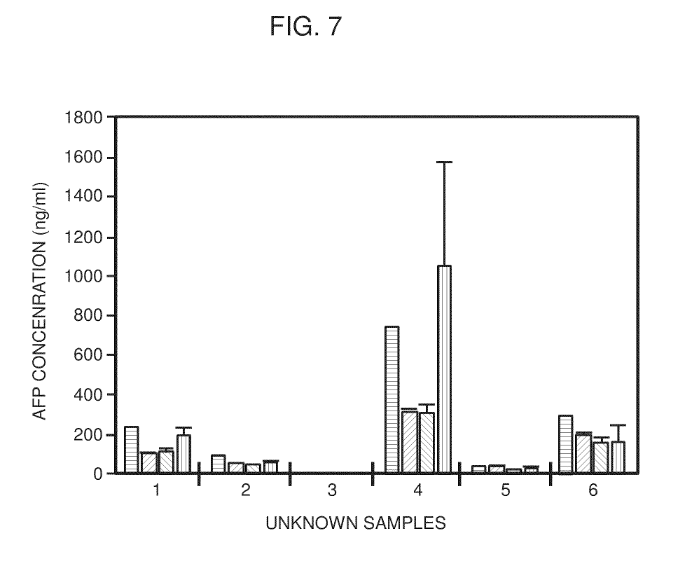

To further evaluate this approach, different amounts of AFP were spiked into human serum, and these samples were then labeled with Alexa Fluor 488 TFP Ester. In either calibration curve or standard addition protocols, the standard concentration should be close to the sample concentration for optimal accuracy and precision. However, in POC screening the AFP concentration is initially unknown. Because the action threshold for serum AFP is 20 ng/mL,^{15, 29} Standard concentrations were set to 5, 10 and 20 ng/mL in a protocol for optimal precision in the diagnostic range. The AFP concentrations measured in the microdevices using both calibration curve and standard addition methods were compared with values measured by a commercial ELISA kit (FIG. 7). In general, both calibration curve and standard addition results matched ELISA results well (FIG. 7 and Table 1). Because the AFP standard concentrations were optimized for the 20 ng/mL diagnostic threshold, higher AFP concentrations (>50 ng/mL) had lower accuracy and precision; however, a POC assay that reports a concentration well above the action level would require more thorough subsequent clinical analysis.

Although these microdevices have been designed for AFP analysis, this approach is not limited to just AFP. These microchips could be easily adapted for detection of other biomarkers by simply immobilizing different antibodies in the affinity column. Moreover, it should be possible to attach multiple antibodies targeting different analytes to the same column, allowing multiplexed, simultaneous biomarker detection. This system shows great promise for rapid quantitation of biomarkers in a POC setting, which should be of considerable value in early stage disease diagnosis.

### Example II

### Characterization of Affinity Columns

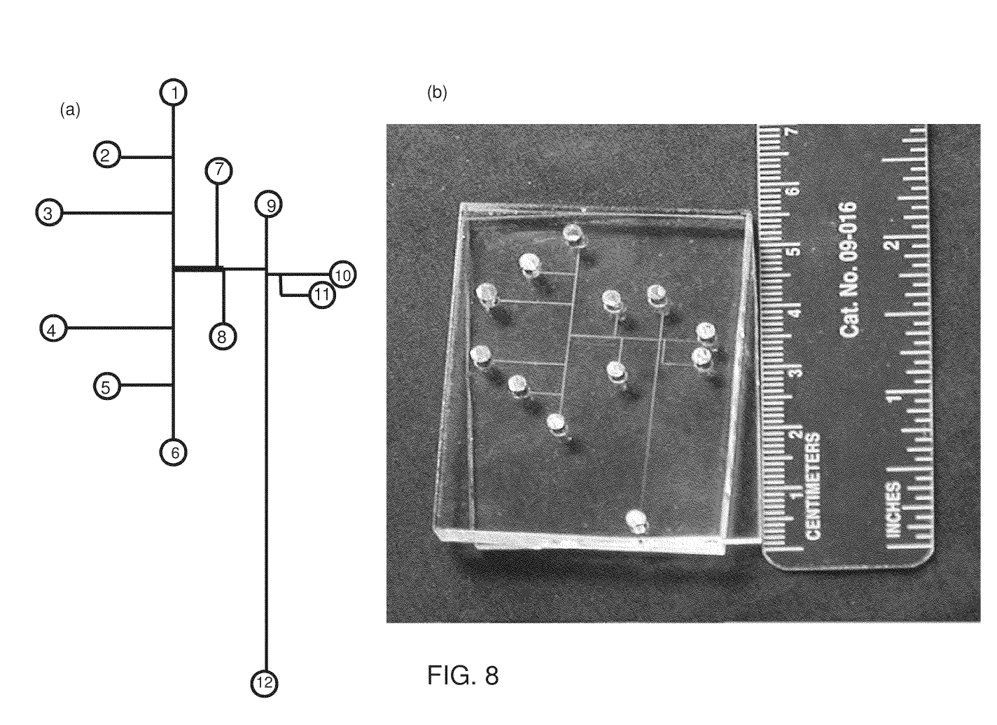

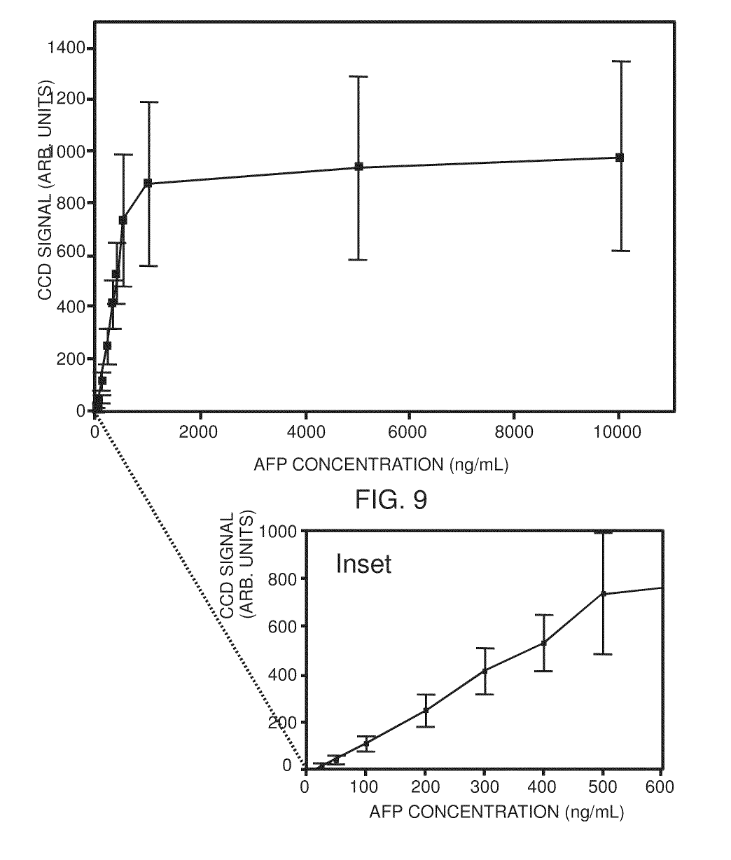

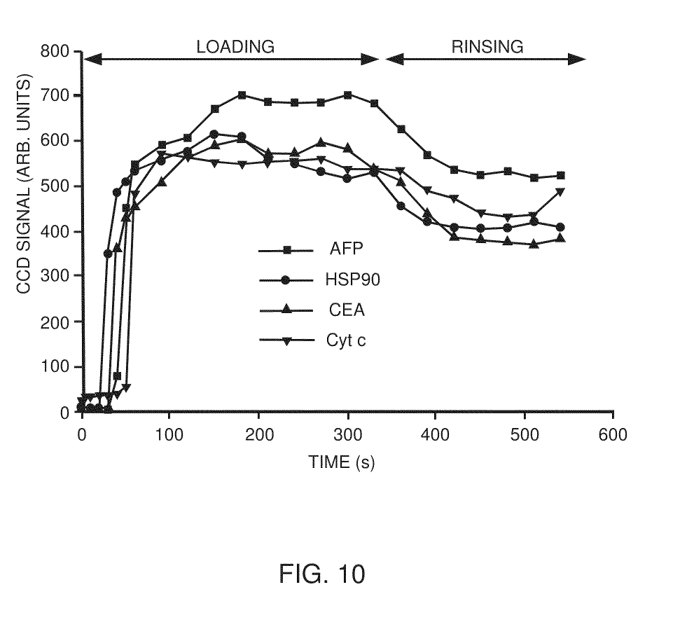

The fluorescence signal on affinity columns for certain exemplary microdevices (Shown in FIG. 8), as a function of AFP concentration is shown in FIG. 9. The relationship between CCD signal and AFP concentration was linear up to ˜500 ng/mL, and the signal approached a plateau at 1 μg/mL. Above ˜1 μg/mL AFP, the antibody sites were all occupied with fluorescently labeled AFP (column saturation), such that the fluorescence signal did not change with further AFP concentration increases. Thus, after loading ˜1 μg/mL of a target protein on the affinity column and washing off unbound material, the maximum amount of retained antigen can be monitored, as shown in FIG. 10. During the rinsing step, the fluorescence signal decreased by ˜15% due to the removal of some unbound protein. Importantly, the signal remained stable after this initial decline during rinsing, indicating strong interaction between antigens and antibodies. In addition, the fluorescence signals of all four proteins were in the same range after rinsing, indicating that the derivatization reaction had little bias toward any of the four antibodies that were used.

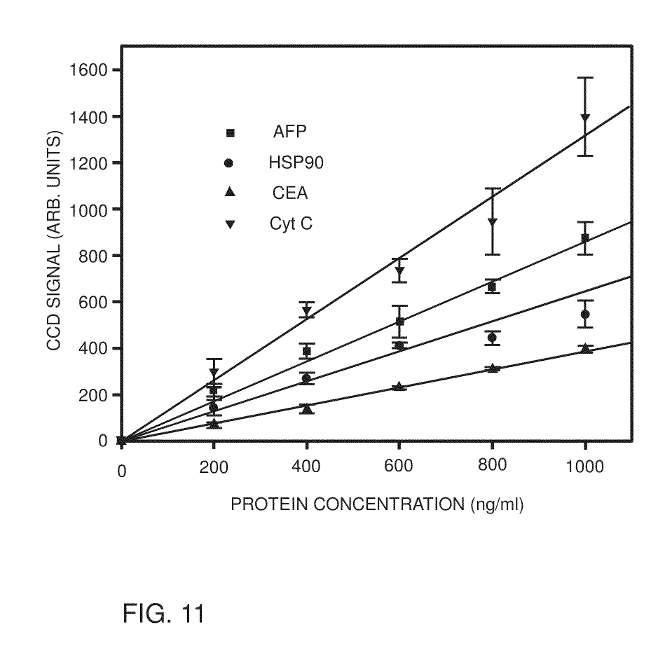

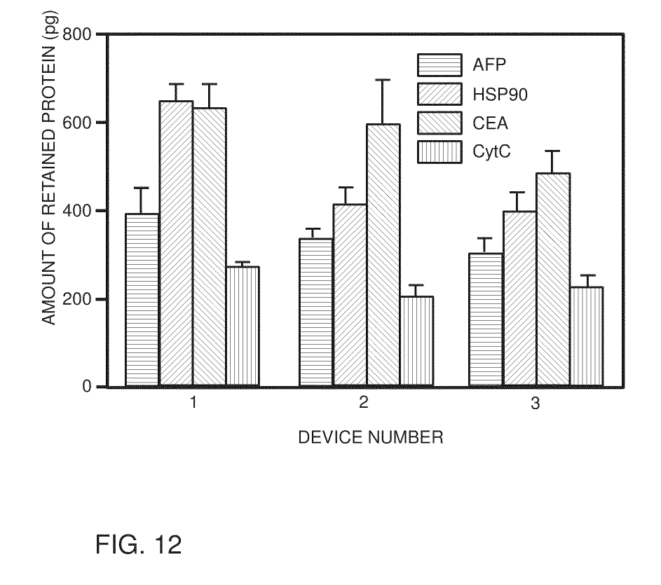

Calibration curves relating fluorescence signal and standard protein concentration were generated in FIG. 11 to convert the CCD signal into the effective concentration of fluorescently labeled protein attached to the column at saturation. For all four proteins, the CCD signal had a linear relationship with protein concentration (R²>0.95). Based on the CCD signal during the rinsing step (FIG. 10) and the 600-nL column volume, the amounts of retained proteins on the affinity column were determined (FIG. 12). The retained protein amounts were all in the range of 0.2 to 0.7 ng, and were also consistent from chip to chip, indicating that immunoaffinity extraction is not affected adversely by multiplexing antibodies on the column. Assuming the antigen-antibody interaction occurs with a 1:1 molar ratio, the average amounts of immobilized anti-AFP, anti-CEA, anti-CytC, and anti-HSP90 were 5, 3, 13, and 5 fmol, respectively (˜10 nmol/L). the channel wall coated affinity columns have a lower density of immobilized antibodies than high surface area, packed porous glass-bead columns (˜5 μmol/L).⁵⁷ Since only microliter or smaller volumes of sample are loaded on the affinity columns, the present binding capacity is not a serious issue for trace (<μg/mL) biomarker analysis. In addition, the density of binding sites in these devices can be easily increased by using a porous material as the solid support. These results demonstrate that affinity columns with four antibodies can be integrated reproducibly in microdevices with good functionality.

Separation of a Model Protein Mixture

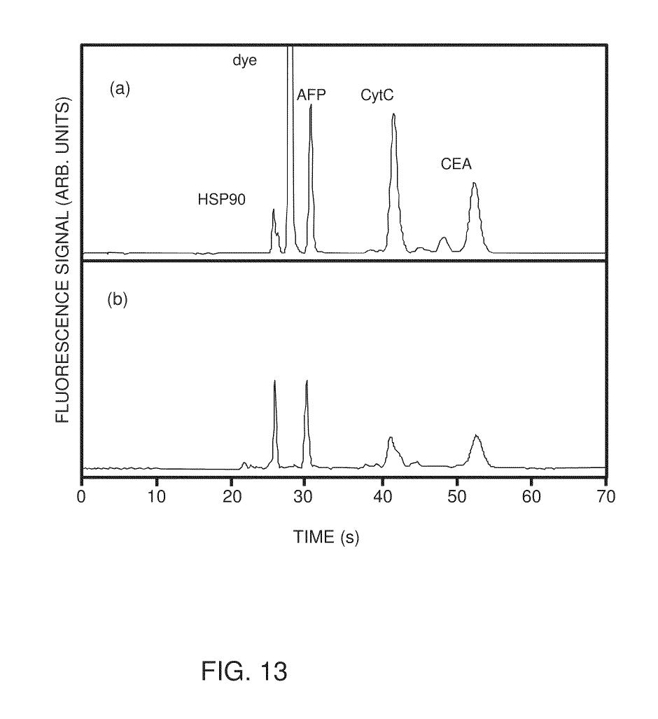

To demonstrate the feasibility of integrated microchip immunoaffinity extraction and CE for multiple biomarker analysis, a mixture of Alexa Fluor 488-labeled AFP, CytC, HSP90 and CEA at 1 μg/mL each in carbonate buffer was analyzed. Five baseline-resolved peaks, including a significant fluorescent dye peak, were observed when this mixture was analyzed by standard microchip CE (without affinity extraction), as shown in FIG. 13a. On the other hand, FIG. 13b shows the electropherogram after this mixture was loaded on an affinity column having the requisite antibodies and then separated by microchip CE after rinsing and elution/injection. With on-chip affinity purification, the dye peak was essentially eliminated (over 10,000-fold reduction), while the four biomarker peaks remained. These results indicate that the integrated microdevices can selectively retain and analyze targeted compounds in samples.

Multiplexed Biomarker Quantitation in Human Serum

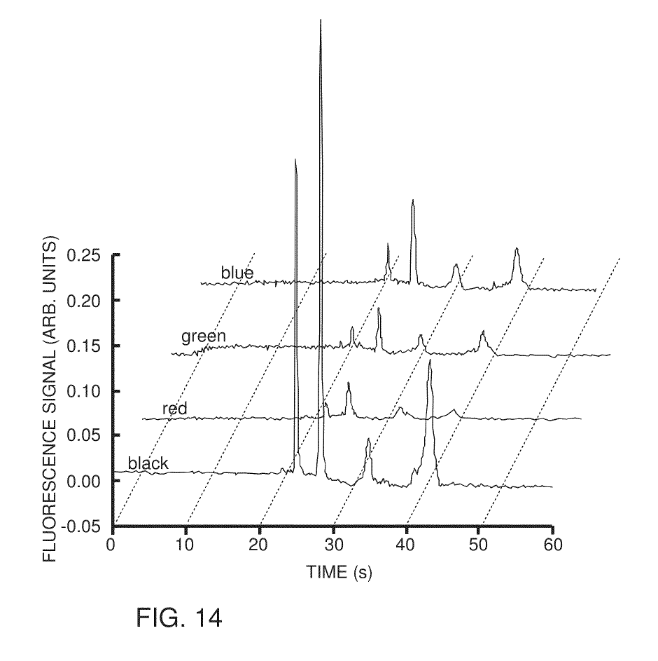

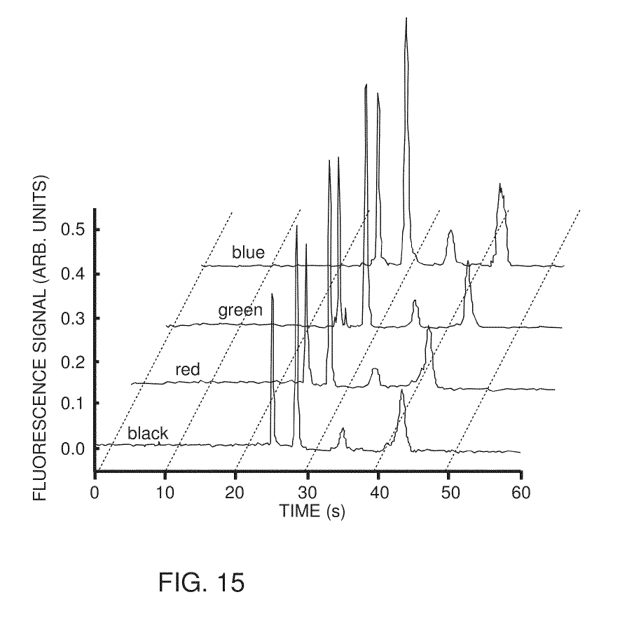

To assess the ability of the present approach to quantify biomarkers in real samples, a series of human blood serum specimens were analyzed that had been spiked with four proteins and fluorescently tagged with Alexa Fluor 488 TFP Ester. Spiked biomarker concentrations in human serum were determined in the integrated affinity extraction and microchip CE devices using either a linear calibration curve (FIG. 14) or the standard addition method (FIG. 15). In FIG. 14, the peak heights of standards increased proportionally going from 5 ng/mL to 20 ng/mL, and the peak heights increased with spiked protein concentration in FIG. 15. In all electropherograms after on-chip affinity purification, only four clean baseline-resolved protein peaks were observed, indicating the efficacy of the multiplexed immunoaffinity extraction column. Four spiked human blood serum samples were tested, and the calibration curve and standard addition results overall matched the known spiked concentrations well (Table 3). In general, the standard deviations for the calibration curve were smaller than those for standard addition; quantitation by standard addition involves extrapolation, which may partially explain the higher standard deviations. Because the serum matrix in the affinity purification step was eliminated, the results were similar for the calibration curve compared to standard addition, which is most effective in complex mixtures.

This approach could be easily extended up to ˜10 biomarker detection by simply immobilizing more antibodies on the affinity column. The surface area of the open channel affinity column (i.e., column saturation) could be an obstacle to scaling to tens of biomarkers, although it is noted that the column saturation level is a factor of at least 25 above the diagnostic threshold for the exemplary markers. Furthermore, the binding capacity could be raised by increasing the surface area of columns (e.g., using a monolith material as the solid support). For more than ˜10 components, the peak capacity in the present device design could be an issue, but a longer folded separation channel⁵⁸ (e.g. 8-cm length) could increase peak capacity to ˜30. Peak capacity could also be raised through spectral multiplexing, wherein several distinct fluorescent labels are used on different proteins. Thus, higher-level multiplexing should be able to significantly increase the number of biomarkers that can be quantified.

To make a real point-of-care (POC) assay, the laser-induced fluorescence (LIF) system and power supplies would need to be miniaturized. A shoebox-size LIF package has been successfully demonstrated for microchip CE analysis of DNA, indicating strong potential to miniaturize the platform for POC applications.⁵⁹ In addition, post-column labeling could be used to decrease the labeling time and reduce operator intervention.⁴⁶ It is further notes that device throughput could be increased by performing separations in parallel,³³ with multiple extraction and separation units on a single chip. Such integrated capillary array devices would enable either replicate sample analysis or higher-level multiplexing.

### EXPERIMENTAL

### Reagents and Materials

CytC (from bovine heart), CEA (from human fluids), monoclonal anti-AFP antibody (produced in mouse), monoclonal anti-CEA antibody (produced in mouse), anti-CytC antibody (produced in sheep), glycidyl methacrylate (GMA, 97%), poly(ethylene glycol) diacrylate (PEGDA, 575 Da average molecular weight), and 2,2-dimethoxy-2-phenylacetophenone (DMPA, 99%) were purchased from Sigma-Aldrich (St. Louis, Mo.). HSP90 and monoclonal anti-HSP90 antibody (produced in mouse) were obtained from Stressgen (Ann Arbor, Mich.). AFP was from Lee Biosolutions (St. Louis, Mo.). Human blood serum from a healthy male (Sigma-Aldrich) was spiked with different concentrations of AFP, CytC, CEA, and HSP90 in the range of 20 to 250 ng/mL (all above normal clinical levels). These unknown serum samples were then labeled with Alexa Fluor 488 TFP Ester (Invitrogen, Eugene, Oreg.) following an Invitrogen protocol (MP 00143). Briefly, 0.1 mg fluorescent dye was dissolved in 10 μL dimethyl sulfoxide (DMSO), and a 2-μL aliquot of DMSO solution was mixed with 98 μL of spiked human serum. The mixture was left to react in the dark at room temperature for 15 min. For protein standards, a 5-μL aliquot of the DMSO solution containing the fluorescent label was mixed with 0.2 mL of 1 mg/mL protein in 10 mM carbonate buffer (pH 9.0). All solutions were prepared with deionized water (18.3 MΩ-cm) purified by a Barnstead EASYpure UV/UF system (Dubuque, Iowa). Poly(methyl methacrylate) (PMMA, Acrylite FF) was purchased from Cyro Industries (Rockaway, N.J.) and was cut into 4.0×5.5 cm² blanks using a CO₂ laser cutter (VLS2.30, Universal Laser Systems, Scottsdale, Ariz.) before device fabrication.

Layout and Fabrication of Microfluidic Devices

The device layout (FIG. 8) and fabrication protocol were adapted from that reported earlier.^{47, 60} Briefly, the microchips contained a sample reservoir (1), two SPE processing reservoirs (2-3) for wash buffer and elution solution, respectively; three reservoirs (4-6) having different standard concentrations for quantification; a waste reservoir (8) for the immunoaffinity extraction step; a reservoir (7) for basic solution (5 mM NaOH) to neutralize the acidic elution solution; and three reservoirs (9, 10 and 12) for standard microchip CE separation. The additional reservoir 11 was originally designed to facilitate the integration of a semi-permeable membrane near the injection intersection, but this capability was not utilized in the present experiments. The microchip pattern was transferred to silicon template wafers using photolithography and wet etching.⁶⁰ PMMA substrates (1.5-mm thick) were imprinted by hot embossing against the etched Si templates.⁴⁹ The patterned PMMA was thermally bonded to an unimprinted PMMA substrate (3.0-mm thick, to provide ˜10 μL reservoir volume capacities) with laser-cut holes (2.0-mm diameter). Channel widths were ˜50 μm, except the affinity column which was 100-μm wide, and channel depths were ˜20 μm.

Since PMMA is inert to many chemical reactions, the microchannel surface was coated to form affinity columns. Briefly, a prepolymer mixture containing GMA (˜60%), PEGDA (˜40%), and DMPA (0.5%) was sonicated and then purged with nitrogen for 3 min to remove dissolved oxygen. The degassed mixture was introduced into the affinity microchannel region via reservoir 7, and a ˜3 μm coating of the prepolymer mixture remained on the channel walls after applying vacuum to reservoir 7 and flowing nitrogen (˜50 psi) from reservoir 1. The microchip was covered with an aluminum photomask, placed on a copper plate in an icebath, and exposed to UV light (320-390 nm, 200 mW/cm²) for 5 min. Finally, unpolymerized material was removed via flushing of 2-propanol through the microchip using a syringe pump.

For immobilization on the patterned affinity channel surface, the four antibodies (anti-AFP, anti-CEA, anti-CytC and anti-HSP90) were mixed at 0.5 mg/mL each in 50 mM borate buffer (pH 8.6). The antibody mixture was pipetted into reservoir 8 and the affinity column filled via capillary action. Borate buffer was placed into all other microchip reservoirs to avoid evaporation during reaction. The entire chip was sealed with 3M Scotch tape (St. Paul, Minn.), and the mixture was left to react at 37° C. for 24 h in the dark.⁶¹ After reaction, the device was flushed using 100 mM Tris buffer (pH 8.3) for 0.5 h. This process also blocked any remaining epoxy groups on the column. Finally, the entire chip was rinsed with carbonate buffer (pH 9.1) before use.

LIF Detection Setup

LIF detection was performed on a Nikon Eclipse TE300 inverted optical microscope equipped with a photomultiplier tube (PMT) detector (Hamamatsu, Bridgewater, N.J.) and CCD camera (Coolsnap HQ, Roper Scientific, Sarasota, Fla.). The LIF detection system and data collection setup have been described previously.^{49,60} CCD images were collected at 10 Hz and analyzed using V++ Precision Digital Imaging software (Auckland, New Zealand). The sampling rate for PMT detection was 20 Hz.

Characterization of Affinity Columns

To estimate the saturation point of affinity columns, different concentrations of fluorescently labeled AFP were loaded for 5 min by applying 400 V at reservoir 8 and 0 V at reservoir 1. Then, unbound AFP was rinsed off the affinity column with PBS buffer for 3 min with 400 V applied to reservoir 8 while grounding reservoir 2. The fluorescence signal on the affinity column was monitored via CCD during the loading and rinsing processes (FIG. 10).

For each analyte, standards of different concentrations were loaded into a microchannel, and fluorescence signal versus protein concentration plots were generated. These calibration curves provided the relationship between CCD signal and the concentration of fluorescently labeled protein in the column in FIG. 11. To determine the amount of immobilized antibodies on the affinity column, 1 μg/mL biomarker standards were loaded on the column with 400 V between reservoirs 1 and 8 for 330 s to saturate all active antibody sites, and the column was washed with Tris buffer using 400 V between reservoirs 2 and 8 for 210 s to remove unbound material. CCD images of the affinity column were recorded at 10 s intervals for the first 60 s, and then 30 s intervals for the remaining time during the loading and washing processes. The CCD signal after washing corresponded to column saturation with antigen; this signal was converted into the equivalent antigen concentration in the column based on the obtained calibration curves. From the column volume of 600 nL, the mass of each protein bound on-chip at saturation was determined. Then, assuming the antigen-antibody interaction occurred with a 1:1 molar ratio, the quantity of immobilized antibodies on the column was determined.

Immunoaffinity Extraction and Electrophoretic Separation

The operation of the integrated microchips and the data analysis were adapted from previous work.⁴⁷ To demonstrate proof-of-principle of multiplexed operation, a mixture of fluorescently labeled AFP, CytC, CEA and HSP90 in buffer was compared before and after microchip immunoaffinity extraction. A double-T microchip layout³¹ was used to directly separate the mixture (without immunoaffinity extraction). The mixture was then pipetted onto an integrated microdevice, loaded for 5 min on the affinity column, rinsed for 5 min, eluted through the injection intersection for 45 s, and then separated by microchip CE.

For calibration curve quantitation, each standard solution containing all four proteins was loaded on the affinity column (5 min), rinsed with PBS buffer (5 min), eluted through the injection intersection for 1 min with phosphate buffer (pH 2.1), and separated by microchip CE, by applying a sequence of potentials to the various reservoirs for all steps.⁴⁷ The sample was analyzed by loading it on the affinity column, rinsing, eluting/injecting and separating the same as for the standards. The peak heights from each standard electropherogram were plotted against the series of known protein concentrations, and linear regression was used to fit a line to the data. The concentration of each component in the sample was calculated from its peak height in the electropherogram and the linear fit equation.

For standard addition quantification, sample was first analyzed the same way as for the calibration curve. Next, sample was loaded on the affinity column for 5 min, followed by loading of the first standard mixture for 5 min; the rinsing, elution/injection and microchip CE separation steps were then carried out as before. This same set of processes was repeated to spike the other two standards into the sample and analyze them.⁴⁸ A linear fit was generated from the peak heights in the electropherograms of the unknown sample and of the sample spiked with standards, plotted against the standard concentrations spiked into the sample. The concentration of each protein was calculated from the intercept and slope of this line.

### CONCLUSION FOR THIS EXAMPLE

Sample pretreatment, cleanup, and quantitation are essential in biomarker analysis in complex media. Affinity purification columns with four different antibodies were prepared in polymer microfluidic devices. The amounts of antibodies immobilized on the columns were consistent from chip to chip, and comparable, low femtomole amounts of each of the four antibodies were attached to the columns. Analysis of four proteins in buffer solution demonstrated that multiplexed immunoaffinity columns could selectively extract the desired species for subsequent CE analysis. With spiked human blood serum samples, four proteins in the ng/mL range were simultaneously quantified using both calibration curves and standard addition. In general, the calibration curve and standard addition results were close to the known spiked concentrations. These microdevices provide an excellent platform for fast, integrated and automated biomarker quantitation. Furthermore, the present system could be expanded to ˜30 biomarker quantitation by immobilizing additional different antibodies on the affinity column, in conjunction with using porous materials for the solid support to improve binding capacity, and longer separation channels as well as spectral multiplexing to raise peak capacity. Importantly, with improvements in engineering and miniaturization, a straightforward POC instrument for multiple biomarker quantitation could result.

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

TABLE 1
Results from the integrated microfluidic AFP assay (the number
that follows the ± sign is the standard deviation).

| | Spiked | | Calibration | Standard |
|---|---|---|---|---|
| | AFP | ELISA | curve | Addition |
| | (ng/mL) | (ng/mL) | (ng/mL) | (ng/mL) |
| unknown 1 | 250 | 110.4 ± 2.7 | 126 ± 6.8 | 198 ± 41 |
| unknown 2 | 100 | 55 ± 2.1 | 52 ± 1.1 | 64 ± 8.8 |
| unknown 3 | 0 | 2.8 ± 2.0 | 4.1 ± 0.9 | 4.6 ± 0.9 |
| unknown 4 | 750 | 323.6 ± 6.7 | 313 ± 41 | 1050 ± 520 |
| unknown 5 | 50 | 49.3 ± 2.0 | 29.4 ± 0.1 | 33.2 ± 2.7 |
| unknown 6 | 300 | 205.1 ± 4.3 | 165 ± 25 | 169 ± 82 |

TABLE 2
Properties of the cancer biomarkers detected.

| | | Normal level | Action threshold |
|---|---|---|---|
| Biomarker | Clinical use | (ng/mL) | (ng/mL) |
| AFP⁵³ | liver cancer | <10 | 20 |
| | marker | | |
| CEA⁵⁴ | colorectal | <5 | 20 |
| | cancer marker | | |
| Cytochrome | prognostic | <0.5 | 25 |
| C (CytC)⁵⁵ | marker during | | |
| | cancer therapy | | |
| Heat shock | many oncogenic | n/a | Overexpression |
| protein 90 | proteins are | | (no action threshold) |
| (HSP90)⁵⁶ | HSP90 clients | | |

TABLE 3
Results from a blinded study with the integrated
microfluidic biomarkers assay chip for spiked human
serum samples (all concentrations are ng/mL).

| | | Concentration | | | Standard deviation | |
|---|---|---|---|---|---|---|
| | | | Cali- | | Cali- | |
| | Sample | | bration | Standard | bration | Standard |
| Analyte | number | Spiked | curve | addition | curve | addition |
| HSP90 | 1 | 110 | 116 | 87 | 7 | 7 |
| | 2 | 183 | 200 | 140 | 13 | 94 |
| | 3 | 219 | 206 | 201 | 13 | 31 |
| | 4 | 58 | 73 | 60 | 4 | 12 |
| AFP | 1 | 116 | 106 | 128 | 7 | 5 |
| | 2 | 140 | 136 | 166 | 10 | 35 |
| | 3 | 37 | 27 | 50 | 2 | 13 |
| | 4 | 70 | 63 | 92 | 4 | 12 |
| CytC | 1 | 200 | 152 | 156 | 25 | 37 |
| | 2 | 53 | 38 | 22 | 5 | 3 |
| | 3 | 106 | 104 | 142 | 16 | 42 |
| | 4 | 160 | 118 | 128 | 19 | 27 |
| CEA | 1 | 27 | 38 | 42 | 2 | 8 |
| | 2 | 50 | 60 | 50 | 4 | 7 |
| | 3 | 83 | 95 | 131 | 6 | 21 |
| | 4 | 100 | 118 | 136 | 8 | 8 |

1. A method for determining concentration of one or more target compounds in a sample solution containing one or more nontarget compounds;
(a) passing the solution through an affinity column, the affinity column having a surface with immobilizing sites for the target compound, so that as the sample is passed through the column target compounds are immobilized on the surface, allowing nontarget compounds to remain in solution as the solution is passed through and out from the column;
(b) eluting the target compounds by passing an eluting buffer solution through the affinity column that reverses the immobilization of the target compounds to produce a target solution containing target compounds;
(c) passing the target solution through a separation column coupled with a detector to produce a quantitative measure of concentration for each of the target compounds.

2. The method of claim 1 wherein the detector determines the quantitative measure by electrophoresis by detecting peaks related to the concentration of the target compounds.

3. The method of claim 1 wherein there are one or more target compounds.

4. The method of claim 1 wherein the target compounds include organic compounds.

5. The method of claim 1 wherein the target compounds include inorganic compounds.

6. The method of claim 1 wherein the solution, eluting buffer are passed by means of one or more of electrically driven systems, pressure driven systems, and electrophoresis systems.

7. The method of claim 1 wherein the detector is based upon a system for detecting fluorescently tagged target molecules of target compounds, optical system, an electrochemical system, chemiluminescence system, or an absorbance system.

8. A method as in claim 1 wherein steps (a) to (c) are applied to multiple solution samples, and quantitative measures of concentration are compared to determine relative concentrations of target compounds.

9. A method as in claim 1 wherein steps (a) to (c) are applied respectively to (1) an unknown sample solution containing unknown amounts of the target compounds, (2) at least one calibration solutions having known amounts of the target compounds, and using a the quantitative measures of concentration for the unknown sample and the calibration samples to determine the concentration of the target compounds in the unknown sample solution using a calibration curve.

10. A method as in claim 1 wherein the quantitative measures are peak heights determined by the detector.

11. A method as in claim 1 wherein steps (a) to (c) are applied respectively to (1) an unknown sample solution containing unknown amounts of the target compounds, (2) at least one standard addition samples having known amounts of the target organic compound added to the unknown sample solution,
and using a the quantitative measures of concentration for the unknown sample and the calibration samples to determine the concentration of the target compounds in the unknown sample solution using a standard addition curve.

12. A method as in claim 1 wherein there are at least two sample solutions.

13. The method of claim 1 wherein the target compound is an antigen and the immobilizing sites include antibodies to the antigen,

14. The method of claim 1 wherein the immobilizing sites are aptamers.

15. The method of claim 1 wherein the target compound is a guest and the immobilizing sites include hosts in a host-guest complex.

16. The method of claim 1 wherein there is more than one target compound in the sample solution.

17. The method of claim 1 wherein there is one target compound in the sample solution.

18. An integrated microdevice comprising:
an affinity column in the form of a microchannel having a surface with immobilizing sites;
structure including channels for selectively directing multiple sample solutions from multiple sample solution sources to and through the affinity column;
the immobilizing sites chosen to immobilize one or more target compounds in a solution passing through the affinity column,
structure including channels for discarding solution passed through the column and after target compounds have been immobilized on the column surface
structure including channels for eluting the affinity column by passing an eluting solution through the column;
structure including one or more separation channels for detecting a quantitative concentrations target compounds in the solution eluted from the affinity column
structure including channels for directing eluted solution from the affinity column to the structure for the detecting structure.

19.-31. (canceled)
